(19) United States
(12) Patent Application Publication
(10) Pub. No.: US 2026/0305981 A1
(43) Pub. Date: Oct. 8, 2026

(54) A METHOD AND A DEVICE FOR CALCULATING A PROBABILITY OF AN ACTUAL FALL EVENT AFTER DETECTING A SUSPECTED FALL EVENT

(71) Applicant: SIGNIFY HOLDING B.V., EINDHOVEN (NL)

(72) Inventors: YING DONG WEI, SHANGHAI (CN); GANG WANG, SHANGHAI (CN); SUFEI LI, SHANGHAI (CN); GONGMING WEI, SHANGHAI (CN); JIA LONG QIU, SHANGHAI (CN)

(21) Appl. No.: 19/164,231

(22) PCT Filed: Mar. 7, 2024
(86) PCT No.: PCT/EP2024/055975

§ 371 Date: Sep. 11, 2025

(30) Foreign Application Priority Data
Mar. 13, 2023 (WO) PCT/CN2023/081164
May 31, 2023 (EP) 23176269.1

(51) Int. Cl. A61B 5/11; A61B 5/00

(52) U.S. Cl. CPC A61B 5/1117; A61B 5/1113; A61B 5/7264; A61B 2503/08

(57) ABSTRACT

A method calculating a probability of an actual fall event after detecting a suspected fall event is disclosed. The method is performed by a processor and comprises the steps of: obtaining sensor data on a person over a defined period prior to detecting of the of a head of the person during a number of stages of the defined period, the features comprising a vertical velocity of the head of the person during each stage and a horizontal displacement of the head of the person during each stage; and calculating the probability of an actual fall event based on the generated features.

### TECHNICAL FIELD

The present disclosure generally relates to the field of fall detection, more particularly, to a method and a device for calculating a probability of an actual fall event after detecting a suspected fall event.

### BACKGROUND

One of the healthcare issues faced by most states and regions in this modern society is to take care of more and more senior people, many of whom living alone. A major health risk for vulnerable population including for example the elderly, infirmed, or disabled is injuries caused by incident fall, such as in the bathroom. Fall, which may be defined as an uncontrolled and sudden displacement of the body of a person to the ground or the floor, if left unnoticed, may cause serious health problem or even lead to death.

It is therefore of much interest to the healthcare industry to monitor fall events happening to the vulnerable people groups. Automated fall detection currently plays an important function for most elderly care solutions.

Compared with wearable device based fall detection methods, contactless fall detection by using remote sensors has some unique advantages and therefore are becoming dominant.

Radar sensors, such as Frequency-Modulated Continuous-Wave radar sensors, have been adopted for years by many elderly care solutions to realize remote fall detection. In recent years, 3 dimensional, 3D, Time of Flight, ToF, sensor based fall detection solutions are emerging.

In a fall detection system employing a ranging sensor such as 3D ToF sensor, the sensor monitors a space, such as space within a bathroom to capture in real time positions and postures of a person in the space. If the person falls and lies on the floor, his or her posture being perceived by the sensor is different from when the person stands or sits.

Different from other sensors such as accelerometer in wearable devices and radar sensors, 3D ToF sensors do not consider the falling behavior/process itself (i.e., before the person lying on the floor) as important for detecting and confirming a fall.

As the 3D ToF sensor based fall detection does not rely on falling behaviors, which may be very different from person to person and from situation to situation, to detect fall events, the 3D ToF sensor based fall detection system can largely reduce false negative (missing detection) and false positive/alarm.

On the other hand, 3D ToF sensors, especially low-resolution ones, can hardly distinguish a lying person who fell from a lying person who is repairing the toilet/drainpipe or person-alike bulky objects such as a pile of clothes being thrown on the floor, for example before being putting into the washing machine. These will trigger false alarm if not handled properly.

In consideration of the above, it is desirable that a probability of an actual fall event can be calculated when a suspected fall event is detected by the fall detection system, so as to reduce false alarms by a fall detection system, especially by ToF sensors based fall detection systems.

### SUMMARY

In a first aspect of the present disclosure, there is presented a method for calculating a probability of an actual fall event after detecting a suspected fall event, the method performed by a processor and comprising the steps of:
obtaining sensor data on a person over a defined period prior to detecting the suspected fall event, upon detection of the suspected fall event;
generating, from the obtained sensor data, features related to movement of a head of the person during a number of stages of the defined period, the features comprising a vertical velocity of the head of the person during each stage and a horizontal displacement of the head of the person during each stage; and
calculating the probability of an actual fall event based on the generated features.

The present disclosure is based on the insight that the probability of an actual fall event happening to a person can be determined based on features related to movement of a head of the person during a defined period before detecting the suspected fall event. The features related to the movement of the head of the person during the falling procedure comprises a vertical velocity of the head of the person and a horizontal displacement of the head of the person during the falling procedure leading to the suspected fall event.

The method is performed after a suspected fall event is detected. Once the suspected fall event is detected, sensor data on the person over a defined period prior to detecting the suspected fall event is obtained. Then the features related to movement of the head of the person in a number of stages of the defined period are generated from the obtained sensor data. Thereafter, such features are used to calculate the probability of an actual fall event.

The method is especially advantageous for fall detection system that determines fall events based on a fallen state of the person. It combines the detection of a potentially fallen person, that is, a lying person, and reviewing a pre-lying process, after the detection of a lying person.

It thereby provides more reliable results as to the probability of the suspected fall event being an actual fall event, reducing undesired false positive. Subsequent decisions such as informing care givers may also be made more reliably.

In an example of the present disclosure, detection of the suspected fall event comprises detecting a person-like object lying on a floor based on sensor data.

As indicated above, the method is especially advantages for fall detection based on a lying state of a person. This is generally realized by detecting, based on the sensor data, a person-like object lying on a floor. A person-like object may be detected as for example reflection from a few zones of the sensor together having a shape similar to a person and having a short distance from the floor. Such detection may be easily made by a ranging sensor.

In an example of the present disclosure, the obtaining step comprises obtaining a number of consecutive measurements taken by a sensor prior to detecting the suspected fall event, the number of consecutive measurements starts prior to or at a moment of a measurement having a highest head position of the person.

This allows features related to the movement of the person during the whole falling procedure to be generated, obtained or derived from the measurements. As a result, a more reliable probability of an actual fall being detected will be obtained. Features related to the movement of the person during the whole falling procedure can be generated at any time point of each stage, such as at the start of each stage, at the end of each stage or at the middle of each stage; or can be averaged values of each whole stage.

In an example of the present disclosure, the generating step comprises:
obtaining a trajectory of the head of the person from the highest head position to a lowest head position based on the obtained sensor data;
dividing the obtained trajectory of the head of the person into a number of stages based on heights;
calculating the vertical velocity of the head of the person during each stage and the horizontal displacement of the head of the person during each stage.

Division of the falling procedure into a number of stages makes it possible to decide the status or movement of the person during the fall procedure in a more accurate way, as the features comprising the vertical velocity and the horizontal displacement of the head of the person in each stage. Variation in the features therefore may be taken into consideration, which is helpful for obtaining a probability of an actual fall which is closer to the real situation.

In an example of the present disclosure, the highest head position is determined as a highest moving point of the person, when the highest moving point is above a threshold value, and the lowest head position is a farthest moving point from feet of the person when the highest moving point of the head of the person is below the threshold value, the feet of the person being parts of the person which are closest to a floor.

This division of the trajectory of the head of the person may be based on the height as described here. That is, the highest and lowest height value of the trajectory are used as end points, and the trajectory are evenly divided in the height direction into several stages.

In another implementation, absolute highest point, such as for example 1.8 meter and lowest point(e.g., 0 meter) are set and the height between the highest and lowest point is divided into several stages.

In an example of the present disclosure, the vertical velocity of the head of the person during each stage comprises one or more of a vertical velocity of the head of the person at any time point of the each stage, e.g. at the start of each stage, at the end of each stage or at the middle of each stage, an average vertical velocity of the head of the person during the each stage, and a weighted vertical velocity of the head of the person during the each stage.

Depending on the expected accuracy, different vertical velocity may be calculated and used to calculate the probability of an actual fall event.

In an example of the present disclosure, the generating step further comprising generating a cross section of the person at each stage.

The cross section may be represented as a number of zones of a sensor occupied by the person. This is also a useful feature for deciding if the suspected fall event is an actual one. It can be used in combination with the vertical velocity and the horizontal displacement of the head of the person to calculate the probability of an actual fall event in a more accurate way.

In an example of the present disclosure, the sensor data is obtained by a ranging sensor, generating a cross section of the person at each stage comprises generating a number of zones of the ranging sensor occupied by the person at the end of each stage, or at any time point of each stage.

In an example of the present disclosure, the calculating step comprises obtaining the probability of an actual fall event from an Artificial Intelligence, AI, classifier by inputting the generated features into the AI classifier.

The principle behind determining the probability of an actual fall from the obtained features is that the features, including the vertical speed, the horizontal displacement of the head of the person, and optionally the horizontal cross section size of the person, in different stages together are closely associated with actual activity types of the person.

A computer implemented algorithm may be used to perform the inference from the feature values to the actual activity type (a fall or not). The algorithm is based on setting thresholds for the various features and arriving at a result by collectively considering the comparison result between the different features and the respective thresholds. Any combination of the three features could be used. For simplicity, in one embodiment, only the vertical velocity and cross section area are used at each stage. As an alternative, we could simply compare these two features at the last stage with threshold values.

For example, when three features related to the movement of the persona and five stages of the height are used, in total fifteen numbers or values will be obtained for the features. These 15 numbers vary a lot when activities of the person change. The determination of a fall depends on both the numbers' absolute values, comparison of the values to the thresholds and the relationship between them.

As an alternative, the method may be implemented using an AI classifier. The AI classifier is trained with tagged dataset. The AI classifier is used to fit the threshold criteria on the numbers or values of the features and get the result. This provides a more efficient way of deriving the probability of an actual fall based on the obtained features and related values.

In an example of the present disclosure, different generated features are assigned different weights.

It can be contemplated that different motion, posture and activity of a person are represented by different features of the person. Therefore, in deciding the probability of an actual fall event, contribution from the different features may be considered differently, which is realized by assigning different weights to the different features. This also helps to improve the accuracy of the calculation of the probability of an actual fall event.

In a specific example of the present disclosure, higher weights are assigned to the vertical velocity of the head of the person as later stages.

This is designed based on the consideration that for an actual fall the person's head picks up speed during the whole falling procedure until the head touches the floor. Therefore the vertical velocity of the head of the person at later stages are considered as a more dominant factor related to an actual fall and thus given a higher weight(s).

In an embodiment of the present disclosure, the method further comprising confirming that an actual fall has happened when the probability is higher than a threshold value.

Such a further step can be easily realized by performing a comparison operation, which is straightforward in terms of implementation and consumes little computational resource.

In an embodiment of the present disclosure, the sensor data is obtained by a Time of Flight, ToF, sensor.

A ToF sensor readily available on the market is a good choice for collecting the sensor data and can be used to obtain the sensor data needed for the method of the present disclosure.

A second aspect of the present disclosure provides an electronic device configured to perform the method according to the first aspect of the present disclosure.

A third aspect of the present disclosure provides a computer program product, comprising a computer readable storage medium storing instructions which, when executed on at least one processor, cause said at least one processor to carry out the method according to the first aspect of the present disclosure.

The above mentioned and other features and advantages of the disclosure will be best understood from the following description referring to the attached drawings. In the drawings, like reference numerals denote identical parts or parts performing an identical or comparable function or operation.

### BRIEF DESCRIPTION OF THE DRAWINGS

FIGS. 1a and 1b respectively illustrate a target perceived by a ToF sensor at a standing position and at a lying-down position.

FIG. 2 schematically illustrates a fall detection system in accordance with the present disclosure.

FIG. 3 schematically illustrates, in a flow chart type diagram, an embodiment of a method of calculating a probability of an actual fall event after detecting a suspected fall event in accordance with the present disclosure.

FIG. 4(a) and 4(b) schematically and exemplarily illustrate a person in a space perceived by a ToF sensor and a trajectory of the head of the person obtained from ToF sensor data.

FIG. 5 schematically illustrates a trajectory of the head of the person being divided into several stages and exemplary features generated for stage 4.

FIG. 6 schematically illustrates an AI classifier used in the present disclosure.

### DETAILED DESCRIPTION

Embodiments contemplated by the present disclosure will now be described in more detail with reference to the accompanying drawings. The disclosed subject matter should not be construed as limited to only the embodiments set forth herein. Rather, the illustrated embodiments are provided by way of example to convey the scope of the subject matter to those skilled in the art.

Throughout the description, the terms “target”, “user” and “subject” are used interchangeably.

As described in the background section, a ranging sensor such as a 3D ToF sensor used in a fall detection system monitors a space in its field of view to capture in real time the position and posture of a person in the space.

Specifically, the ToF sensor measures a distance between the sensor and an object within its field of view by emitting a burst of light, usually in the form of infrared light, and measuring a time it takes for the light to bounce back off the object and return to the sensor.

For a ToF sensor with high enough resolution, lights reflected back from different parts of the body may be used by the sensor to construct a depth map that can represent a 3D shape of the person.

In contrast, when the ToF sensor is of low-resolution, the sensor may not tell from the depth map constructed form lights reflected back to the sensor from the person's body whether the detected object is a person or something else such as a furniture.

However, the ToF sensor is capable of distinguishing a person lying on a floor and a person who is standing or sitting, as posture of the fallen person as being perceived by the ToF sensor is different from when the person stands or sits.

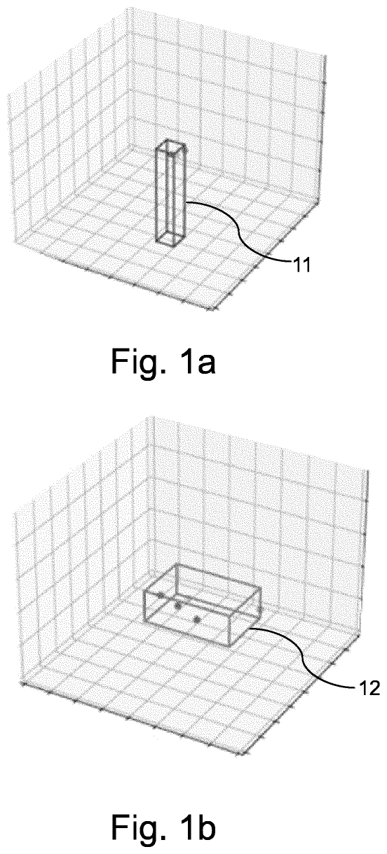

Referring to Figures la and 1b, which respectively illustrate a target perceived by a ToF sensor at a standing position and at a lying-down position, a column 11 as illustrated in Figure la represents a standing person from the view of a low-resolution, for example 8×8, 3D ToF sensor, while a column 12 as illustrated in FIG. 1b is how the person is perceived by the 3D ToF sensor when the person falls and lies on the floor.

Based on the above, the 3D ToF sensor may be used to detect a fall event based reflection from an object which is captured by a few zones of the sensor together having a shape similar to a person and having a short distance from the floor.

However, such reflection may also be caused by a purposed lain down person who is repairing the toilet/drainpipe or bulky (person-alike) objects such as pile of clothes being thrown on the floor before putting into the washing machine. These will trigger false alarm if not handled properly.

The present disclosure proposes a solution to distinguish an actual fall from intentionally lying down on the floor, which may be detected by the ranging sensor as a suspected call. The method of the present helps to reduce false alarm of a low-resolution ToF sensor-based fall detection system.

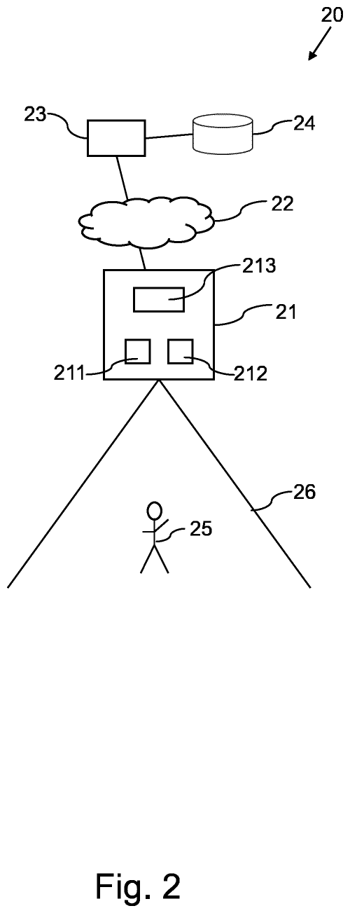

FIG. 2 schematically illustrates a fall detection system 20 in accordance with the present disclosure. The fall detection system comprises a ranging sensor such as a TOF sensor 21, which is connected via a network 22 such as the internet or a home network to a processing device 23. The processing device 23 may comprise or is communicatively connected to a storage device 24.

The sensor 21 has an emitter 211 that emits a signal such an infrared signal and a receiver 212 that senses signals reflected from an object, here a person 25, in a detection range or field of view 26 of the sensor 21. The sensor 21 may also include an integrated processor 213 that controls operation of the sensor 21 and calculates a time of flight of the emitted signal, i.e., the time between a time the emitter 211 emitted the signal and the time when the receiver 212 receives the reflected signal.

An algorithm for detecting motion or presence in the detection range of the sensor 21 based on data collected by the sensor 21 may run on the processor 213 of the sensor 21 or on the processing device 23 connected to the sensor 21 via the network 22. Such an algorithm may be stored for example in the storage device 24 or an internal storage device, not shown, in the sensor 21.

The sensor 21 of the fall detection system 20 may be mounted on a ceiling of for example a washroom, so as to monitor motion and posture of a person in the washroom without capturing any privacy information.

When a person enters a space having the sensor installed therein and gets detected by the sensor 21 via a presence detection algorithm, the sensor 21 starts to monitor the motion and posture of the person. In the meantime, the sensor 21 keeps recording its measurements over a pre-defined duration or time period for future use.

In an implementation, a buffer of for example 60 second is used to store the data. Every time a new measurement is obtained, the buffer will get rid of the oldest measurement data (if the buffer is full) and store the new one.

For the purpose of reducing a size of recorded data, the sensor may only record the measurements of zones covering the person.

The time period may be for example 20 seconds. Such measurement or sensor data allows the present disclosure to review a fall procedure resulted in a suspected fall event, once the suspected fall event is detected.

The sensor 21 keeps monitoring the motion and posture of the person. When the sensor 21 detects a possible or suspected fall of the person, i.e., by identifying a person-alike object lying on the floor, the fall detection system will start a perform a method of calculating a probability of an actual fall event, so as to confirm whether the suspected fall event is an actual one.

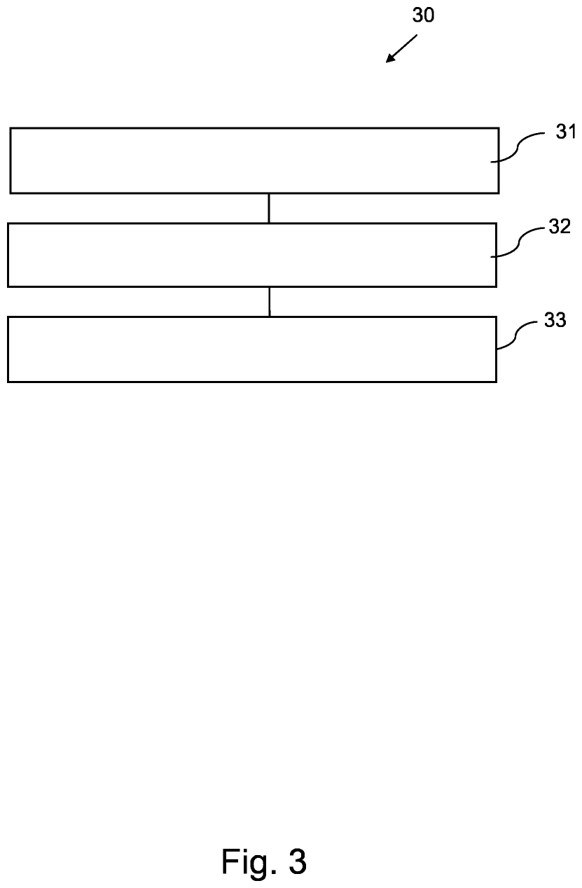

FIG. 3 schematically illustrates, in a flow chart type diagram, an embodiment of a method 30 of calculating a probability of an actual fall event after detecting a suspected fall event in accordance with the present disclosure.

The method may be performed by a processor, such as the processor 213 integrated into the sensor 21, or a processor 23 which may be deployed remotely or locally to the space having the sensor installed therein. The processor 23 may be comprised in for example a computing device including a computer, a mobile phone or other computing devices having sufficient computing capacity to perform the method of the present disclosure.

At step 31, upon detection of the suspected fall event, sensor data on a person over a defined period prior to detecting of the suspected fall event is obtained. This may comprise for example a remote processing device receiving the sensor data over the defined period from the sensor, or a processor of the sensor retrieves the sensor data saved locally in the sensor.

At step 32, features related to movement of the head of the person during the defined period are generated from the obtained sensor data.

The defined period is a period comprising the fall procedure resulting in the detected suspected fall event. For the purpose of improving the accuracy of the calculation result regarding the probability of an actual fall, as will be described next, the falling procedure is divided into several stages based on height.

The generated features comprise a vertical velocity of the head of the person during each stage and a horizontal displacement of the head of the person during each stage. The generated features may further comprise a horizontal cross section of the person during each stage.

At step 33, the generated features are used to calculate the probability of an actual fall event.

The above procedure will be described in detail in the following with reference to an example of calculating the probability of an actual fall event based on sensor data acquired by a ToF sensor.

When a suspected fall event is detected by the ToF sensor, for example by detecting a person-like object lying on a floor based on the sensor data as discussed above, ToF sensor measurements or sensor data over a period of for example 20 second are obtained. As a real fall procedure happens out of control and abruptly, it normally takes a very short time such as within one or two seconds. It is needed that the obtained measurements start prior to or at a moment of a measurement having a highest head position of the person.

From the obtained measurements, positions of the person's head are extracted, a trajectory of the head of the person is hence generated. The head position is determined by using the highest moving point as the head top when the highest moving point is above a threshold (e.g., 1 meter), and using the farthest moving point from the feet when the highest moving point is below the threshold. The feet are the part of the human body closest to the floor.

Then a trajectory of the head is generated based on head positions. The trajectory starts when the head is at the highest position and ends when the head is at the lowest position. The trajectory is further divided into several stages based on the height of the head.

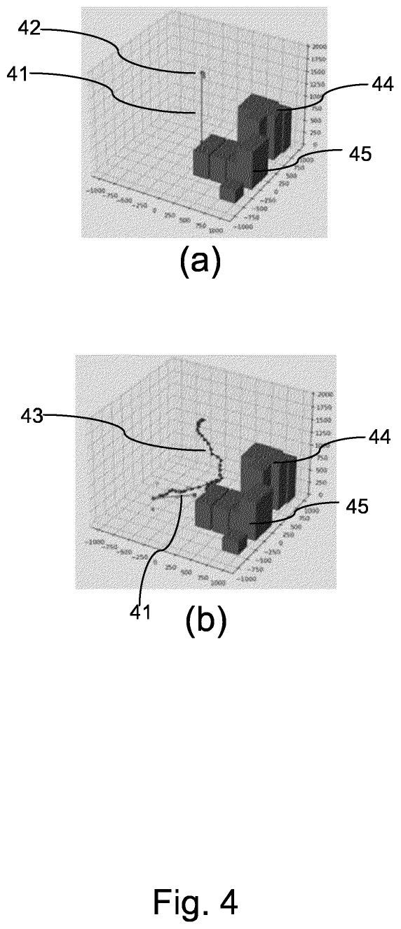

FIG. 4(a) and 4(b) schematically and exemplarily illustrate a person in a space perceived by the ToF sensor and a trajectory of the head of the person obtained from the sensor data.

Referring to FIG. 4(a), a straight line segment 41 represents a standing person, with a small dot 42 on a top end of the straight line segment 41 representing the head of the person.

When the position of the head extracted from each measurement of the defined period is plotted against the 3D floor map representing the space where the fall event takes place, a curve 43 as illustrated in FIG. 4(b) is obtained. The curve 43 is the moving trajectory of the head of the person during the falling procedure, which starts at a moment when the head is at the highest position and ends when the head is at the lowest position. In FIG. 4(a) and 4(b), reference numerals 44 and 45 represent other objects that are present in the space where the fall event takes place, such as a basin or a toilet.

Next, the trajectory is divided into several stages based on height, thereby dividing the procedure when the head is moving form the highest position to the lowest position into several stages. The height here can be an absolute height from the ground or floor, or the relative height in the process of a fall.

For each stage, several features with high correlation with human body posture and activity are generated. These features include a vertical velocity of the head, a horizontal displacement of the head, and optionally a horizontal cross-section of the person.

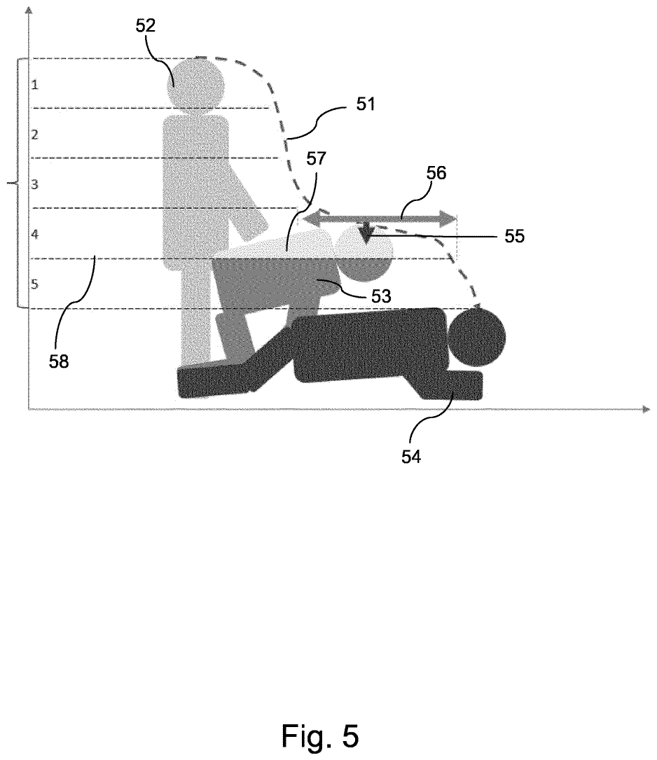

Referring to FIG. 5, a trajectory 51 of the head of the person, illustrating the head moving from the highest position to the lowest position and indicated by a dashed line, is divided into five stages. For better understanding of extracting features from the measurement data in each stage, a human with three different postures are also illustrated. Specifically, a person in standing position 52 is illustrated in a light grey shade, a person in crouching position 53 is illustrated in a dark grey shade, and a lying person 54 is shown in black shade.

A vertical height of the trajectory 51 is divided evenly into five stages. As an alternative, division of the trajectory 51 may be based on a height decided by an absolute highest point, such as for example 1.8 meter, and an absolute lowest point(e.g., 0 meter). Such a height covering trajectory 51 is set and is divided into several stages, thereby also dividing the trajectory 51 into several stages.

The measurements or sensor data are then used to generate features with high correlation with human body posture and activity. Such features include: the vertical velocity of the head of the person and the horizontal displacement of the head of the person during each stage. A horizontal cross-section of the person during each stage may also be generated.

In FIG. 5 the above features of stage 4 are illustrated. The horizontal axis of FIG. 5 is used to indicate a horizontal direction, and the vertical direction indicates the height direction.

The vertical velocity of the head of the person during each stage may comprise one or more of a vertical velocity of the head of the person at any time point of the each stage, an average vertical velocity of the head of the person during the each stage, and a weighted vertical velocity of the head of the person during the each stage. For the purpose of simplifying the subsequent calculation procedure, the vertical velocity of the head at the end of each stage may be conveniently used, which is illustrated in FIG. 5 as a downward black arrow 55.

The horizontal displacement of the head during each stage refers to a distance travelled by the head in each stage. In FIG. 5 an exemplary horizontal displacement of the head during stage 4 is indicated by a left-right double arrow 56.

The horizontal cross-section of the person in each stage is decided when the head is of the person is at the end of each stage. It can be determined based on a number of zones of the ToF sensor occupied by the person. In FIG. 5, a part of the human body above a horizontal line 58 between stage 4 and 5 is the horizontal cross-section of the person at the end of stage.

These features are generated in different head height, since the ToF ranging sensor has a high resolution in the height direction.

Following that, a probability of the detected suspected fall event being an actual fall event will be calculated based on the above generated features as these features are closely associated with actual activity types of the person.

Setting threshold is done in the training process. And in inferencing, the features are inputted into a generated model to calculate the probability. This calculation includes comparison between features and threshold, comparison between different features, and a weighted combination of features and the comparison results.

To be specific, inference is made from different feature values to the actual activity type. A mathematical model including the thresholds for the various features are generated in the training process based on the training data. The inferencing is done by arriving at a result by collectively considering the comparison results between the different features and the respective thresholds. The probability of the detected suspected fall event being an actual fall event is thereby obtained.

Such a procedure can be carried out using an artificial intelligence, AI, classifier by inputting the generated features of all the stages thereinto. Optionally, different weights might be assigned to different features. The weights are determined in training process and then used in inferencing. For example, the vertical velocity at later stages (e.g., stage 4 and 5) can be given a greater weight. The AI classifier is pre-trained with tagged datasets.

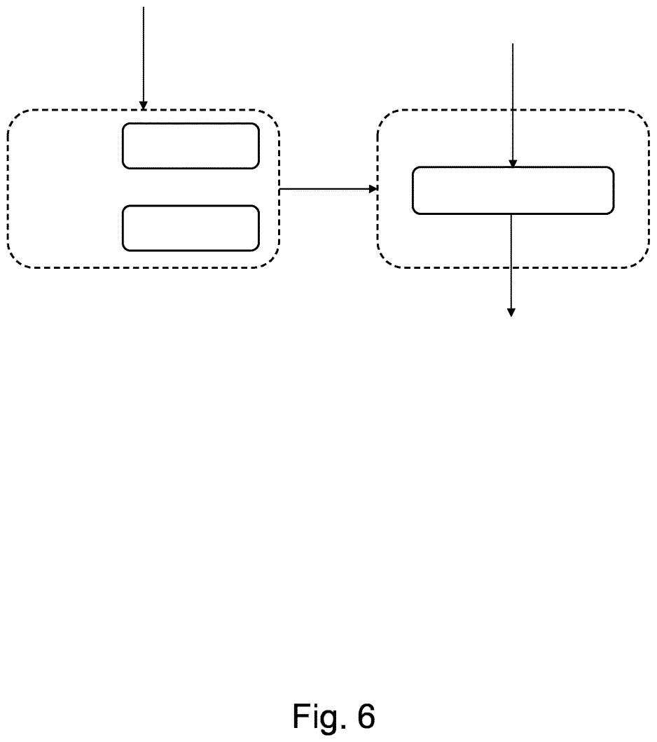

FIG. 6 schematically illustrates an AI classifier used in the present disclosure. The AI classifier can be one or a combination of several algorithms such as XGBoost, LightGBM, and neural network.

The AI classifier select a flexible ensemble model. A first ensembled model XGBoost/LightGBM is based on decision tree and a second neural network is based on logistics regression, both of which are quite flexible and can fit complex mappings.

These two different kinds of basic models give complementary result, and then they are weighted combined to get a better result. This kind of ensemble model is suitable for the present disclosure, since the sensor data used in the present disclosure are space locations related and variations of the sensor data over time. The problem solved by the present disclosure is not a time series problem since it doesn't happen regularly nor there are some definite relations between states in time before and after.

The classifier can be deployed on a local device, such as a luminaire which integrates the ToF sensor, or in a cloud server. If the AI classifier is deployed in the cloud server, the features will be generated in edge devices including the ToF sensor of the fall detection system as illustrated in FIG. 1 and transferred to the server over the network. These data can be further collected and used to train the AI model iteratively and enhance its performance.

The AI classifier generates an actual fall probability rate and output it.

In a test case performed using the AI classifier described above, 20 cases of actual fall and 20 cases of intentionally lying down were collected and randomly partitioned into a training set (80%) and a test set (other 20%) to train an XGBoost classifier. This classifier has no errors for the training set and the test set under different partition of the training/test set.

This solution combines the method of detection a lying person and reviewing the pre-lying process after the detection of a lying person. It helps to decrease the false positive of the ToF ranging sensor-based fall detection system.

The computation power requirement of the system is not large due to the following factures, firstly, with the ToF ranging data, a person-alike object on the floor could be easily detected, secondly, the fall confirmation process, including the AI classification, only works when a person-alike object is detected.

The present disclosure is not limited to the examples as disclosed above, and can be modified and enhanced by those skilled in the art beyond the scope of the present disclosure as disclosed in the appended claims without having to apply inventive skills and for use in any data communication, data exchange and data processing environment, system or network.

1. A method for calculating a probability of an actual fall event after detecting a suspected fall event, the method performed by a processor and comprising the steps of:
obtaining sensor data on a person over a defined period prior to detecting of the suspected fall event, upon detection of the suspected fall event;
generating, from the obtained sensor data, features related to movement of a head of the person during a number of stages of the defined period, the features comprising a vertical velocity of the head of the person during each stage and a horizontal displacement of the head of the person during each stage; and
calculating the probability of an actual fall event based on the generated features;
wherein the sensor data obtained by the processor is obtained by a 3D Time of Flight, ToF, sensor.

2. The method according to claim 1, wherein detection of the suspected fall event comprises detecting a person-like object lying on a floor based on sensor data.

3. The method according to claim 1, wherein the obtaining step comprises obtaining a number of consecutive measurements taken by a sensor prior to detecting the suspected fall event, the number of consecutive measurements starts prior to or at a moment of a measurement having a highest head position of the person.

4. The method according to claim 3, wherein the generating step comprises:
obtaining a trajectory of the head of the person from the highest head position to a lowest head position based on the obtained sensor data;
dividing the obtained trajectory of the head of the person into a number of stages based on heights;
calculating the vertical velocity of the head of the person during each stage and the horizontal displacement of the head of the person during each stage.

5. The method according to claim 3, wherein the highest head position is determined as a highest moving point of the head of the person, when the highest moving point is above a threshold value, and the lowest head position is a farthest moving point from feet of the person when the highest moving point of the head of the person is below the threshold value, the feet of the person being parts of the person which are closest to a floor.

6. The method according to claim 1, where the vertical velocity of the head of the person during each stage comprises a vertical velocity of the head of the person at any time point of the each stage, an average vertical velocity of the head of the person during the each stage, and a weighted vertical velocity of the head of the person during the each stage.

7. The method according to claim 1, wherein the generating step further comprising generating a cross section of the person at each stage.

8. The method according to claim 7, wherein the sensor data is obtained by a ranging sensor, generating a cross section of the person at each stage comprises generating a number of zones of the ranging sensor occupied by the person at the end of each stage.

9. The method according to claim 1, wherein the calculating step comprises obtaining the probability of an actual fall event from an Artificial Intelligence, AI, classifier by inputting the generated features into the AI classifier.

10. The method according to claim 9, wherein different generated features are assigned different weights.

11. The method according to claim 10, wherein higher weights are assigned to the vertical velocity of the head of the person as later stages.

12. The method according to claim 1, further comprising confirming that an actual fall has happened when the probability is higher than a threshold value.

13. An electronic device configured to perform the method according to claim 1.

14. A computer program product, comprising a computer readable storage medium storing instructions which, when executed on at least one processor, cause said at least one processor to carry out the method according to claim 1.
